(19) United States
(12) Patent Application Publication
(10) Pub. No.: US 2026/0354915 A1
(43) Pub. Date: Nov. 12, 2026

(54) SYSTEMS AND METHODS FOR AUTOMATED ROBOTIC ENDOVASCULAR MEDICAL DEVICE MANIPULATION

(71) Applicant: Siemens Healthineers Endovascular Robotics Inc., Newton, MA (US)

(72) Inventors: Young-Ho Kim, West Windsor, NJ (US); Eric Lluch, Nürnberg (DE); Mehmet Akif Gulsun, Princeton, NJ (US); Ankur Kapoor, Plainsboro, NJ (US); Florin-Cristian Ghesu, Baiersdorf (DE); Dorin Comaniciu, Princeton, NJ (US)

(21) Appl. No.: 19/130,693

(22) PCT Filed: Feb. 16, 2024
(86) PCT No.: PCT/EP2024/053993

§ 371 Date: May 16, 2025

Related U.S. Application Data
(60) Provisional application No. 63/485,582, filed on Feb. 17, 2023.

(51) Int. Cl. A61B 34/32; A61B 34/20; A61B 34/30; A61M 25/09; G16H 40/63

(52) U.S. Cl. CPC A61B 34/32; G16H 40/63; A61B 2034/2065; A61B 2034/301; A61M 25/09041

(57) ABSTRACT

One or more example embodiments provides a method comprising obtaining, by a trained system, at least one movement command for an intervention device of a robotic surgical system to a location within a patient; determining, by the trained system, at least one actuator command for the robotic surgical system associated with the at least one movement command based on the movement command and an estimated current position of the intervention device; and applying the actuator command to the robotic surgical system.

### CROSS-REFERENCE TO RELATED APPLICATIONS

The present application claims priority under 35 U.S.C. § 119 (e) to U.S. provisional patent application No. 63/485,582, titled AI-based Agents for Automated Robotic Endovascular Guidewire Manipulation, filed Feb. 17, 2023, the entire contents of which are hereby incorporated by reference.

### FIELD

One or more example embodiments relate to systems and methods for automated robotic endovascular medical device manipulation.

### BACKGROUND

Endovascular guidewire manipulation is used for minimally-invasive clinical applications. Percutaneous Coronary Intervention (PCI) is used to open narrowed coronary arteries and restore arterial blood flow to heart tissue, mechanical thrombectomy techniques are used for acute ischemic stroke (AIS) to remove blood clots from the brain veins, and transjugular intrahepatic portosystemic shunt (TIPS) is used for liver portal hypertension use a special needle and position a wire between the portal vein through the liver.

These procedures commonly use 3D vessel geometries from 3D CTA (Computed Tomography Angiography) images.

### SUMMARY

The scope of protection sought for various example embodiments is set out by the independent claims. The example embodiments and/or features, if any, described in this specification that do not fall under the scope of the independent claims are to be interpreted as examples useful for understanding various embodiments.

Independent of the grammatical term usage, individuals with male, female or other gender identities are included within the term.

Unfortunately, conventional X-ray targets and associated components are not suitable for the large currents and/or instantaneous dose rates characteristic of UHDR radiotherapy. For example, if exposed to the high beam currents and/or instantaneous dose rates characteristic of UHDR radiotherapy, conventional X-ray targets may deteriorate and/or be destroyed.

According to at least one example embodiment, a method includes obtaining, by a trained system, at least one movement command for an intervention device of a robotic surgical system to a location within a patient; determining, by the trained system, at least one actuator command for the robotic surgical system associated with the at least one movement command based on the movement command and an estimated current position of the intervention device; and applying the actuator command to the robotic surgical system.

According to at least one example embodiment, the intervention device is a guidewire.

According to at least one example embodiment, the at least one movement command is a desired location within the patient of a tip of the guidewire.

According to at least one example embodiment, the method further includes obtaining an observed position of the guidewire from the robotic surgical system; and determining the estimated current position of the guidewire based on the observed position.

According to at least one example embodiment, the observed position of the intervention device corresponds to an observed position of a proximal portion of the guidewire.

According to at least one example embodiment, the estimated current position corresponds to an estimated position of a tip of the guidewire.

According to at least one example embodiment, the determining determines a series of actuator commands, each actuator command of the series of actuator commands corresponds to a state of the guidewire.

According to at least one example embodiment, the method further includes generating image data displaying the estimated current position of the guidewire within a vessel of the patient.

According to at least one example embodiment, the estimated position of the guidewire is overlaid on a three-dimensional vessel segmentation.

According to at least one example embodiment, the actuator command is at least one of a velocity command or a torque command for the actuator.

According to at least one example embodiment, a trained system includes processing circuitry configured to cause the trained system to obtain at least one movement command for an intervention device of a robotic surgical system to a location within a patient, determine at least one actuator command for the robotic surgical system associated with the at least one movement command based on the movement command and an estimated current position of the intervention device, and apply the actuator command to the robotic surgical system.

According to at least one example embodiment, the intervention device is a guidewire.

According to at least one example embodiment, the at least one movement command is a desired location within the patient of a tip of the guidewire.

According to at least one example embodiment, the processing circuitry is configured to cause the trained system to obtain an observed position of the guidewire from the robotic surgical system; and determine the estimated current position of the guidewire based on the observed position.

According to at least one example embodiment, the observed position of the intervention device corresponds to an observed position of a proximal portion of the guidewire.

According to at least one example embodiment, the estimated current position corresponds to an estimated position of a tip of the guidewire.

According to at least one example embodiment, the processing circuitry is configured to cause the trained system to determine a series of actuator commands, each actuator command of the series of actuator commands corresponding to a state of the guidewire.

According to at least one example embodiment, the processing circuitry is configured to cause the trained system to generate image data displaying the estimated current position of the guidewire within a vessel of the patient.

According to at least one example embodiment, the estimated position of the guidewire is overlaid on a three-dimensional vessel segmentation.

According to at least one example embodiment, the actuator command is at least one of a velocity command or a torque command for the actuator.

According to at least one example embodiment, a trained system includes means for obtaining at least one movement command for an intervention device of a robotic surgical system to a location within a patient; means for determining at least one actuator command for the robotic surgical system associated with the at least one movement command based on the movement command and an estimated current position of the intervention device; and means for applying the actuator command to the robotic surgical system.

### BRIEF DESCRIPTION OF THE DRAWINGS

Example embodiments will become more fully understood from the detailed description given herein below and the accompanying drawings, wherein like elements are represented by like reference numerals, which are given by way of illustration only and thus are not limiting of this disclosure.

FIG. 1 is a perspective view of a robotic procedure system according to one or more example embodiments;

FIG. 2 is a block diagram of a robotic procedure system according to one or more example embodiments;

FIG. 3 is a block diagram of a robotic procedure system depicting various actuating mechanisms according to according to one or more example embodiments;

FIG. 4A illustrates a predictive control system according to one or more example embodiments;

FIG. 4B illustrates a flow chart of generating a physics-based simulation of a guidewire according to one or more example embodiments;

FIG. 4C illustrates a flow chart of generating a new mesh with a desired vessel-like structure to restrict the movement of a portion of a guidewire according to one or more example embodiments;

FIG. 4D illustrates a level set map and a gradient level set map projected onto a surface of a lumen according to one or more example embodiments;

FIGS. 5A-5D illustrates a flow chart of using various images to generate a physics-based simulation of a guidewire according to one or more example embodiments;

FIG. 6A illustrates a training flow diagram according to one or more example embodiments;

FIG. 6B illustrates a 3D vessel geometry with a centerline;

FIG. 7A illustrates a flow chart of an implementation of an AI predictive control system according to one or more example embodiments;

FIG. 7B illustrates a method of operation of the AI predictive control system of FIG. 7A according to one or more example embodiments;

FIG. 8 illustrates a forward agent shown in FIG. 7A according to one or more example embodiments;

FIG. 9 illustrates an inverse agent shown in FIG. 7A according to one or more example embodiments; and

FIG. 10 illustrates an example transformer model used to implement the AI predictive control system according to one or more example embodiments.

It should be noted that these figures are intended to illustrate the general characteristics of methods, structure and/or materials utilized in certain example embodiments and to supplement the written description provided below. These drawings are not, however, to scale and may not precisely reflect the precise structural or performance characteristics of any given embodiment and should not be interpreted as defining or limiting the range of values or properties encompassed by example embodiments. The use of similar or identical reference numbers in the various drawings is intended to indicate the presence of a similar or identical element or feature.

### DETAILED DESCRIPTION

Various example embodiments will now be described more fully with reference to the accompanying drawings in which some example embodiments are shown.

Detailed illustrative embodiments are disclosed herein. However, specific structural and functional details disclosed herein are merely representative for purposes of describing example embodiments. The example embodiments may, however, be embodied in many alternate forms and should not be construed as limited to only the embodiments set forth herein.

It should be understood that there is no intent to limit example embodiments to the particular forms disclosed. On the contrary, example embodiments are to cover all modifications, equivalents, and alternatives falling within the scope of this disclosure. Like numbers refer to like elements throughout the description of the figures.

As discussed herein the terminology “one or more” and “at least one” may be used interchangeably.

It will be appreciated that a number of example embodiments may be used in combination.

Minimally-invasive clinical applications procedures commonly use 3D vessel geometries from 3D CTA (Computed Tomography Angiography) images. During these procedures, the clinician manipulates a wire through the catheter and across the blockage. The clinician only uses X-ray fluoroscopy intermittently to visualize and guide the catheter, guidewire, and other devices (e.g., angioplasty balloons and stents).

Various types of endovascular robot-assisted systems (e.g., Corindus™ TechNIQ™) are being developed to provide efficient positional control of devices, helping clinicians to mitigate therapeutical risks. The primary motions that a clinician can use to control the movement and direction of the wire include rotation and pushing/retracting from the proximal end of the wire outside the insertion point on the patient's body.

As used herein, the direction distal is the direction toward the patient and the direction proximal is the direction away from the patient. The terms up and upper refer to the general direction away from the direction of gravity and the terms bottom, lower and down refer to the general direction of gravity.

While the below example embodiments are described with reference to controlling a guidewire, it should be noted that other endovascular devices may be used.

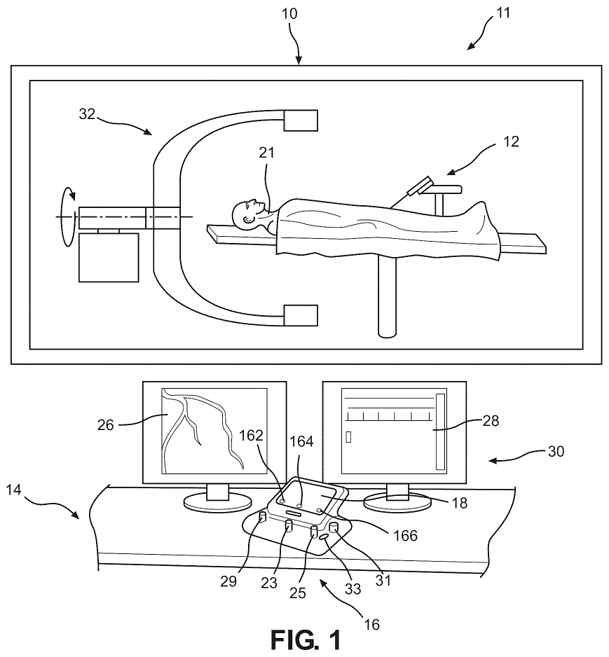

Referring to FIG. 1, a robotic procedure system 10 is shown. Robotic procedure system 10 may be used to perform catheter based medical procedures (e.g., percutaneous intervention procedures). Percutaneous intervention procedures may include diagnostic catheterization procedures during which one or more catheters are used to aid in the diagnosis of a patient's disease. For example, during one embodiment of a catheter based diagnostic procedure, a contrast media is injected into one or more coronary arteries through a catheter and an image of the patient's heart is taken. Percutaneous intervention procedures may also include catheter based therapeutic procedures (e.g., balloon angioplasty, stent placement, treatment of peripheral vascular disease, etc.) during which a catheter is used to treat a disease. It should be noted, however, that one skilled in the art would recognize that, certain specific percutaneous intervention devices or components (e.g., type of guidewire, type of catheter, etc.) will be selected based on the type of procedure that is to be performed. Robotic procedure system 10 is capable of performing any number of catheter based medical procedures with minor adjustments to accommodate the specific percutaneous devices to be used in the procedure. In particular, while the embodiments of robotic procedure system 10 described herein are explained primarily in relation to the diagnosis and/or treatment of coronary disease, robotic procedure system 10 may be used to diagnose and/or treat any type of disease or condition amenable to diagnosis and/or treatment via a catheter based procedure. For example, robotic procedure system 10 may be used for the treatment of hypertension utilizing a radiofrequency emitting catheter to deactivate certain nerves that enervate the kidneys to control hypertension.

Robotic procedure system 10 includes lab unit 11 and workstation 14. Robotic procedure system 10 includes a robotic catheter system, shown as bedside system 12, located within lab unit 11 adjacent patient 21. Generally, bedside system 12 may be equipped with the appropriate percutaneous devices (e.g., guidewires, guide catheters, working catheters, catheter balloons, stents, diagnostic catheters, etc.) or other components (e.g., contrast media, medicine, etc.) to allow the user to perform a catheter based medical procedure. A robotic catheter system, such as bedside system 12, may be any system configured to allow a user to perform a catheter-based medical procedure via a bedside system by operating various controls such as the controls located at workstation 14. Bedside system 12 may include any number and/or combination of components to provide bedside system 12 with the functionality described herein.

Various embodiments of bedside system 12 are described in detail in P.C.T. International Application No. PCT/US2021/070042, filed Nov. 14, 2021, PCT/US2020/041964, filed Jul. 7, 2020, P.C.T. International Application No. PCT/US2009/042720, filed May 4, 2009, the entire contents of each of which are incorporated herein by reference.

In one embodiment, bedside system 12 may be equipped to perform a catheter based diagnostic procedure. In this embodiment, bedside system 12 may be equipped with one or more of a variety of catheters for the delivery of contrast media to the coronary arteries. In one embodiment, bedside system 12 may be equipped with a first catheter shaped to deliver contrast media to the coronary arteries on the left side of the heart, a second catheter shaped to deliver contrast media to the coronary arteries on the right side of the heart, and a third catheter shaped to deliver contrast media into the chambers of the heart.

In another embodiment, bedside system 12 may be equipped to perform a catheter based therapeutic procedure. In this embodiment, bedside system 12 may be equipped with a guide catheter, a guidewire, and a working catheter (e.g., a balloon catheter, a stent delivery catheter, ablation catheter, microcatheter, etc.). In one embodiment, bedside system 12 may be equipped with a working catheter that includes a secondary lumen that is threaded over the guidewire during a procedure. In another embodiment, bedside system 12 may be equipped with an over-the-wire working catheter that includes a central lumen that is threaded over the guidewire during a procedure. In another embodiment, bedside system 12 may be equipped with an intravascular ultrasound (IVUS) catheter. In another embodiment, any of the percutaneous devices of bedside system 12 may be equipped with positional sensors that indicate the position of the component within the body.

Bedside system 12 is in communication with workstation 14, allowing signals generated by the user inputs and/or control system of workstation 14 to be transmitted to bedside system 12 to control the various functions of bedside system 12. Bedside system 12 also may provide feedback signals (e.g., operating conditions, warning signals, error codes, etc.) to workstation 14. Bedside system 12 may be connected to workstation 14 via a communication link 38 that may be a wireless connection, cable connectors, or any other means capable of allowing communication to occur between workstation 14 and bedside system 12.

Workstation 14 includes a user interface 30. User interface 30 includes controls 16. Controls 16 allow the user to control bedside system 12 to perform a catheter based medical procedure. For example, controls 16 may be configured to cause bedside system 12 to perform various tasks using the various percutaneous devices with which bedside system 12 may be equipped (e.g., to advance, retract, or rotate a guidewire, advance, retract, or rotate a working catheter, advance, retract, or rotate a guide catheter, inflate or deflate a balloon located on a catheter, position and/or deploy a stent, inject contrast media into a catheter, inject medicine into a catheter, or to perform any other function that may be performed as part of a catheter based medical procedure, etc.). In some embodiments, one or more of the percutaneous intervention devices may be steerable, and controls 16 may be configured to allow a user to steer one or more steerable percutaneous device. In one such embodiment, bedside system 12 may be equipped with a steerable guide catheter, and controls 16 may also be configured to allow the user located at remote workstation 14 to control the bending of the distal tip of a steerable guide catheter. Further embodiments of catheter system 10 including a steerable guide catheter are disclosed in U.S. U.S. Pat. No. 9,452,277, the entire contents of which are incorporated herein by reference. Another example embodiment of a control system in shown in U.S. Ser. No. 17/812,733, filed Jul. 15, 2022, incorporated by reference herein.

In one embodiment, controls 16 include a touch screen 18, a dedicated guide catheter control 29, a dedicated guidewire control 23, and a dedicated working catheter control 25. In this embodiment, guidewire control 23 is a joystick configured to advance, retract, or rotate a guidewire, working catheter control 25 is a joystick configured to advance, retract, or rotate a working catheter, and guide catheter control 29 is a joystick configured to advance, retract, or rotate a guide catheter. In addition, touch screen 18 may display one or more icons (such as icons 162, 164, and 166) that control movement of one or more percutaneous devices via bedside system 12 or to receive various inputs from the user as discussed below. Controls 16 may also include a balloon or stent control that is configured to inflate or deflate a balloon and/or a stent. Each of the controls may include one or more buttons, joysticks, touch screens, etc., that may be desirable to control the particular component to which the control is dedicated. As discussed in more detail below, robotic procedure system 10 includes a percutaneous device movement algorithm module or movement instruction module 114 that dictates how bedside system 12 responds to a user's manipulation of controls 16 to cause a percutaneous device to move in a particular way.

Controls 16 may include an emergency stop button 31 and a multiplier button 33. When emergency stop button 31 is pushed a relay is triggered to cut the power supply to bedside system 12. Multiplier button 33 acts to increase or decrease the speed at which the associated component is moved in response to a manipulation of guide catheter control 29, guidewire control 23, and working catheter control 25. For example, if operation of guidewire control 23 advances the guidewire at a rate of 1 mm/sec, pushing multiplier button 33 may cause operation of guidewire control 23 to advance the guidewire at a rate of 2 mm/sec. Multiplier button 33 may be a toggle allowing the multiplier effect to be toggled on and off. In another embodiment, multiplier button 33 must be held down by the user to increase the speed of a component during operation of controls 16.

User interface 30 may include a first monitor 26 and a second monitor 28. First monitor 26 and second monitor 28 may be configured to display information or patient specific data to the user located at workstation 14. For example, first monitor 26 and second monitor 28 may be configured to display image data (e.g., x-ray images, MRI images, CT images, ultrasound images, etc.), hemodynamic data (e.g., blood pressure, heart rate, etc.), patient record information (e.g., medical history, age, weight, etc.). In addition, first monitor 26 and second monitor 28 may be configured to display procedure specific information (e.g., duration of procedure, catheter or guidewire position, volume of medicine or contrast agent delivered, etc.). In one embodiment, the user may interact with or select various icons or information displayed on monitors 26 and 28 using a user input device or control (e.g., a mouse). Monitor 26 and monitor 28 may be configured to display information regarding the position and/or bend of the distal tip of a steerable guide catheter. Further, monitor 26 and monitor 28 may be configured to display information to provide the functionalities associated with the various modules of controller 40 discussed below. In another embodiment, user interface 30 includes a single screen of sufficient size to display one or more of the display components and/or touch screen components discussed herein.

Robotic procedure system 10 also includes an imaging system 32 located within lab unit 11. Imaging system 32 may be any medical imaging system that may be used in conjunction with a catheter based medical procedure (e.g., non-digital x-ray, digital x-ray, CT, MRI, ultrasound, etc.). In an exemplary embodiment, imaging system 32 is a digital x-ray imaging device that is in communication with workstation 14. As shown in FIG. 1, imaging system 32 may include a C-arm that allows imaging system 32 to partially or completely rotate around patient 21 in order to obtain images at different angular positions relative to patient 21 (e.g., sagittal views, caudal views, cranio-caudal views, etc.).

Imaging system 32 is configured to take x-ray images of the appropriate area of patient 21 during a particular procedure. For example, imaging system 32 may be configured to take one or more x-ray images of the heart to diagnose a heart condition. Imaging system 32 may also be configured to take one or more x-ray images during a catheter based medical procedure (e.g., real-time images) to assist the user of workstation 14 to properly position a guidewire, guide catheter, working catheter, stent, etc. during the procedure. The image or images may be displayed on first monitor 26 and/or second monitor 28.

In addition, the user of workstation 14 may be able to control the angular position of imaging system 32 relative to the patient to obtain and display various views of the patient's heart on first monitor 26 and/or second monitor 28. Displaying different views at different portions of the procedure may aid the user of workstation 14 properly move and position the percutaneous devices within the 3D geometry of the patient's heart. For example, displaying the proper view during a procedure may allow the user to view a patient's vascular system from the proper angle to ensure that the distal tip of a steerable guide catheter is bent in the proper way to ensure the catheter is moved as intended. In addition, displaying different views at different portions of a procedure may aid the user in selecting the appropriate instruction set of movement instruction module 114 discussed below. In an exemplary embodiment, imaging system 32 may be any 3D imaging modality of the past, present, or future, such as an x-ray based computed tomography (CT) imaging device, a magnetic resonance imaging device, a 3D ultrasound imaging device, etc. In this embodiment, the image of the patient's heart that is displayed during a procedure may be a 3D image. In addition, controls 16 may also be configured to allow the user positioned at workstation 14 to control various functions of imaging system 32 (e.g., image capture, magnification, collimation, c-arm positioning, etc.).

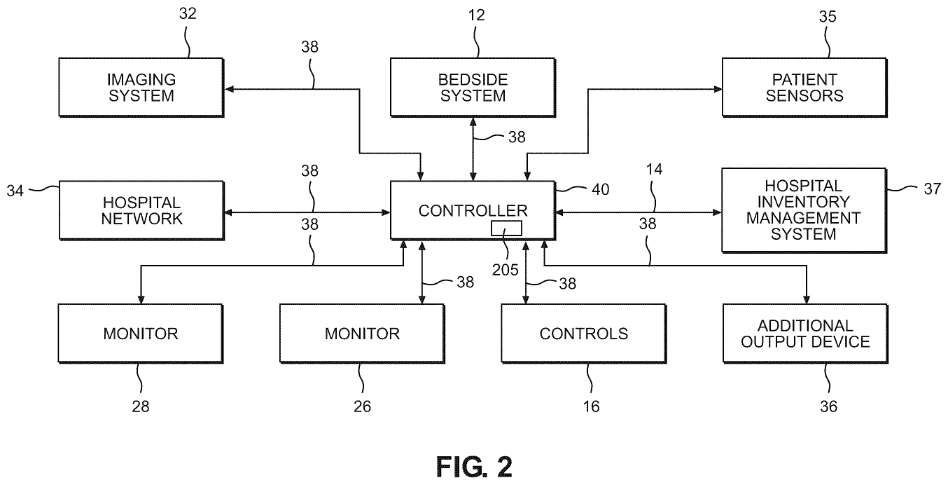

Referring to FIG. 2, a block diagram of robotic procedure system 10 is shown according to an exemplary embodiment. Robotic procedure system 10 may include a control system, shown as controller 40. While a controller 40 is described, it should be understood example embodiments are not limited thereto. For example, processing or control circuitry may be used such as, but not limited to, one or more processors, one or more Central Processing Units (CPUs), one or more controllers, one or more arithmetic logic units (ALUs), one or more digital signal processors (DSPs), one or more microcomputers, one or more field programmable gate arrays (FPGAs), one or more System-on-Chips (SoCs), one or more programmable logic units (PLUS), one or more microprocessors, one or more Application Specific Integrated Circuits (ASICs), or any other device or devices capable of responding to and executing instructions in a defined manner.

As shown in FIG. 2, controller 40 may be part of workstation 14. Controller 40 is in communication with one or more bedside systems 12, controls 16, monitors 26 and 28, imaging system 32, and patient sensors 35 (e.g., electrocardiogram (“ECG”) devices, electroencephalogram (“EEG”) devices, blood pressure monitors, temperature monitors, heart rate monitors, respiratory monitors, etc.). In addition, controller 40 may be in communication with a hospital data management system or hospital network 34, one or more additional output devices 36 (e.g., printer, disk drive, cd/dvd writer, etc.), and a hospital inventory management system 37.

Communication between the various components of robotic procedure system 10 may be accomplished via communication links 38. Communication links 38 may be dedicated wires or wireless connections. Communication links 38 may also represent communication over a network. Robotic procedure system 10 may be connected or configured to include any other systems and/or devices not explicitly shown. For example, robotic procedure system 10 may include IVUS systems, image processing engines, data storage and archive systems, automatic balloon and/or stent inflation systems, contrast media and/or medicine injection systems, medicine tracking and/or logging systems, user logs, encryption systems, systems to restrict access or use of robotic procedure system 10, robotic catheter systems of the past, present, or future, etc. Further embodiments of robotic procedure system 10 including inflation and/or contrast media injection systems are disclosed in U.S. Pat. No. 9,545,497, issued Jan. 17, 2017, which is incorporated herein by reference in its entirety.

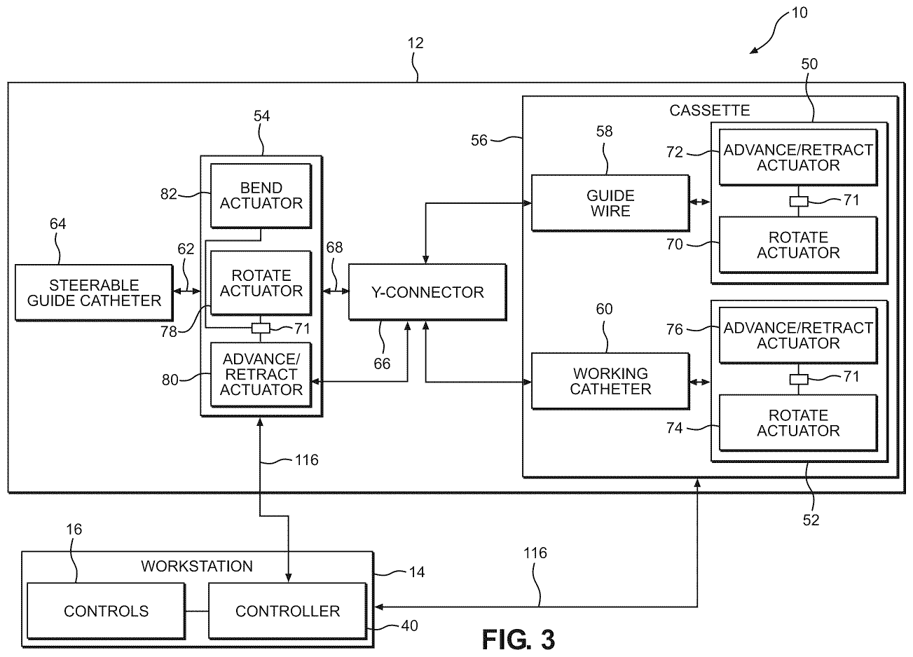

Referring to FIG. 3, a block diagram of an embodiment of robotic procedure system 10 is shown according to an exemplary embodiment. Robotic procedure system 10 may include various actuating mechanisms that move an associated percutaneous device in response to a user's manipulation of controls 16. In the embodiment shown, robotic procedure system 10 includes a guidewire actuating mechanism 50, a working catheter actuating mechanism 52, and a guide catheter actuating mechanism 54. In other embodiments, robotic procedure system 10 may include an actuating mechanism for inflating an angioplasty or stent delivery balloon and an actuating mechanism for delivering contrast agent. In the embodiment shown, guidewire actuating mechanism 50 and working catheter actuating mechanism 52 are incorporated within cassette 56 which is coupled to a base of bedside system 12. Additional embodiments of bedside system 12 and cassette 56 are described in detail in P.C.T. International Application No. PCT/US2021/070042, filed Nov. 14, 2021, P.C.T. International Application No. PCT/US2020/041964, filed Jul. 7, 2020, P.C.T. International Application No. PCT/US2020/041923, filed Jul. 14, 2020, U.S. Pat. No. 8,480,618, issued Jul. 9, 2013, the entire contents of each of which are incorporated herein by reference. Further embodiments of robotic procedure system 10 are described in detail in P.C.T. International Application No. PCT/US2021/070042, filed Nov. 14, 2021, P.C.T. International Application No. PCT/US2020/041964, filed Jul. 7, 2020, P.C.T. International Application No. PCT/US2020/041923, filed Jul. 14, 2020, U.S. Pat. No. 8,790,297, issued Jul. 29, 2014, and in U.S. Pat. No. 9,545,497, issued Jan. 17, 2017, the entire contents of each of which are incorporated herein by reference.

Guidewire actuating mechanism 50 is coupled to guidewire 58 such that guidewire actuating mechanism 50 is able to cause guidewire 58 to advance, retract, and rotate. Working catheter actuating mechanism 52 is coupled to working catheter 60 such that working catheter actuating mechanism 52 is able to cause working catheter 60 to advance, retract, and rotate. Connector 62 couples guide catheter 64 to guide catheter actuating mechanism 54 such that guide catheter actuating mechanism 54 is able to cause guide catheter 64 to advance, retract, and rotate. In various embodiments, guidewire actuating mechanism 50, working catheter actuating mechanism 52, and guide catheter actuating mechanism may each include an engagement structure (e.g., one or more pairs of pinch wheels) suitable for engaging the respective percutaneous device such that the actuating mechanism is able to impart axial and/or rotational movement to the percutaneous device.

A Y-connector 66 is coupled to guide catheter actuating mechanism 54 via connector 68. In various embodiments, connector 68 may be a component separate from both Y-connector 66 and guide catheter actuating mechanism 54. In other embodiments, connector 68 may be part of (e.g., integral with) Y-connector 66 or part of actuating mechanism 54. In the embodiment shown, Y-connector 66 is also connected to cassette 56.

In one embodiment, Y-connector 66 includes a first leg, a second leg, and a third leg. The first leg of the Y-connector is connected to or in communication with the internal lumen of guide catheter 64. The second leg is angled away from the longitudinal axis of guide catheter 64. The second leg provides a port for the injection of fluids (e.g., contrast media, medicine, etc.) into the lumen of guide catheter 64. The third leg of Y-connector 66 is coupled to a cassette 56 and receives both guidewire 58 and working catheter 60. Thus, by this arrangement, guidewire 58 and working catheter 60 are inserted through Y-connector 66 into the internal lumen of guide catheter 64.

Guidewire actuating mechanism 50 includes a rotate actuator 70 and an advance/retract actuator 72. Rotate actuator 70 is configured to cause rotation of guidewire 58 about its longitudinal axis. The guidewire actuating mechanism 50 may further include one or more sensors 71 for measuring the velocity, torque, position and other parameters of the guidewire/actuator. Advance/retract actuator 72 is configured to advance and/or retract guidewire 58 (i.e., to advance and/or retract along the longitudinal axis of the guidewire) within patient 21. Working catheter actuating mechanism 52 includes a rotate actuator 74 and an advance/retract actuator 76. Rotate actuator 74 is configured to cause rotation of working catheter 60 about its longitudinal axis. The working catheter 60 may further include one or more sensors 71 for measuring the velocity, torque, position and other parameters of the guidewire/actuator. Advance/retract actuator 76 is configured to advance and/or retract working catheter 60 (i.e., to advance and/or retract along the longitudinal axis of the working catheter) within patient 21. Guide catheter actuating mechanism 54 includes a rotate actuator 78, an advance/retract actuator 80, and a bend actuator 82. The guide catheter actuating mechanism 54 may further include one or more sensors 71 for measuring the velocity, torque, position and other parameters of the guidewire/actuator. Rotate actuator 78 is configured to cause rotation of guide catheter 64 about its longitudinal axis. Advance/retract actuator 80 is configured to advance and/or retract guide catheter 64 (i.e., to advance and/or retract along the longitudinal axis of the guide catheter) within patient 21. In some embodiments, guide catheter 64 may include one or more bend control elements that allow the user to cause bending of the distal tip of guide catheter 64. In such an embodiment, bend actuator 82 causes the distal tip of guide catheter 64 to bend in response to a user's manipulation of controls 16.

As shown in the block diagram of FIG. 3, controls 16 and controller 40 located at workstation 14 are communicably coupled to various portions of bedside system 12 to allow the user and/or control system to control movement of guidewire 58, working catheter 60 and guide catheter 64 and any other percutaneous devices that bedside system 12 is equipped with. In the embodiment shown, controls 16 and controller 40 are coupled to guide catheter actuating mechanism 54 to allow the user to move guide catheter 64. In addition, controls 16 and controller 40 are coupled to cassette 56 to allow the user to control guidewire 58 via guidewire actuating mechanism 50 and to control working catheter 60 via working catheter actuating mechanism 52. Control signals 116 generated by the controls and controller at workstation 14 are communicated to bedside system 12 to control movement of percutaneous devices discussed herein.

Even with these robotic devices, clinicians passively control guidewires/catheters by relying on limited indirect observation (i.e., 2D partial view of devices, and intermittent updates due to radiation limit) from X-ray fluoroscopy. The observed device states from X-ray fluoroscopy shows a limited view, and are not clear to estimate exact state information, leading to increase overall execution time and not a long-term prediction to guide the human user.

Modeling and controlling the guidewire manipulation in coronary vessels remains challenging because of the complicated interaction between guidewire motions with different physical properties (i.e., loads, coating) and vessel geometries with lumen conditions resulting in a highly nonlinear system.

Wiring a vessel is a challenging problem. Especially, there exist multiple devices (e.g., microcatheter, guidewire, catheter, etc.) for neuro catheterization, which interact with each other in vessels. The devices are controlled by twisting, pushing and, pulling off each device until it arrives at the desired location in vessel, which is highly dependent on the clinicians' experience. During this period, X-ray images are repeatedly acquired in order to visualize the desired catheter path and verify when it has entered the desired vessel, subjecting the clinician to excess radiation.

Current robotic endovascular robotic procedures have the following limitations: decision-making and safety estimation for robotic controls, which previously highly relying on the user's experience, are missing.

Example embodiments provide (1) a scalable learning pipeline to train an AI predictive control system, providing a safety feature for long-term controls; (2) a long-term integrated predictive control for multiple devices in robotic neuro catheterization.

Thus, predictive controls according to one or more example embodiments alleviate the clinician's fatigue and reduce the overall execution time, which is proportional to X-ray exposure time.

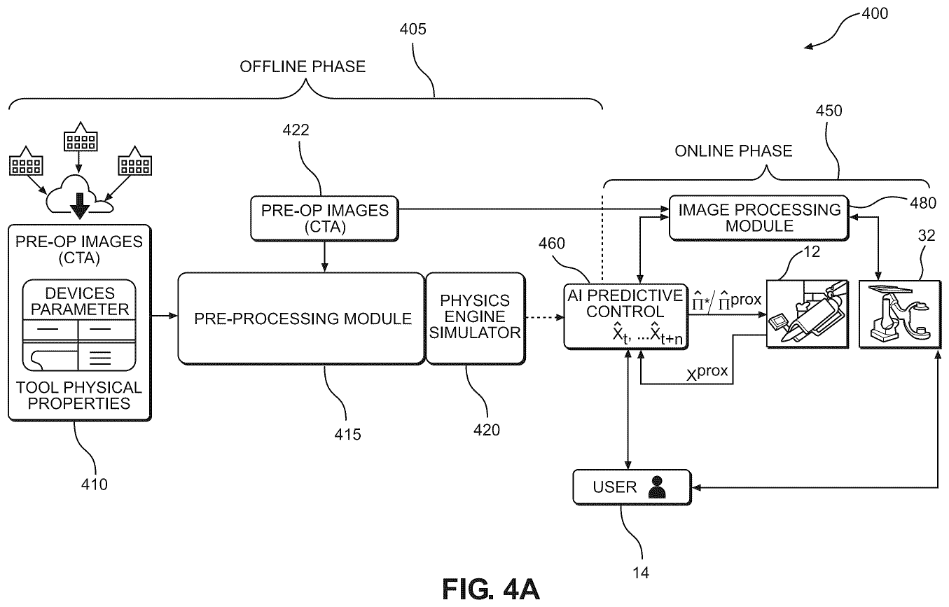

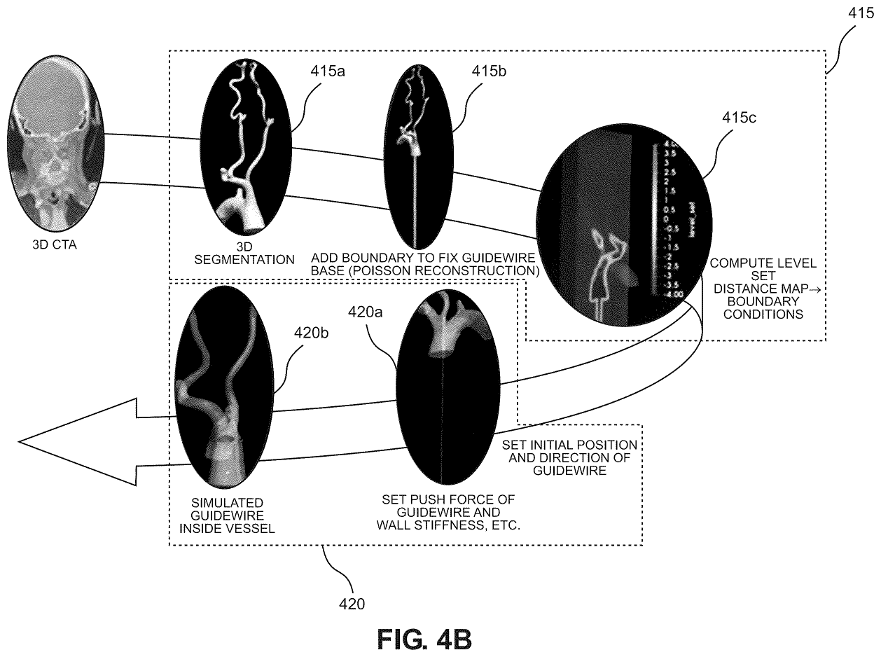

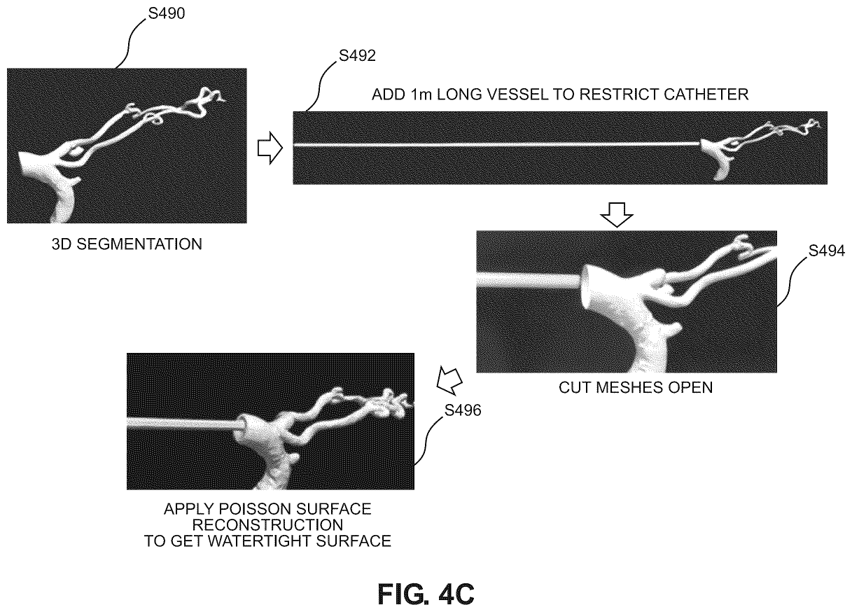

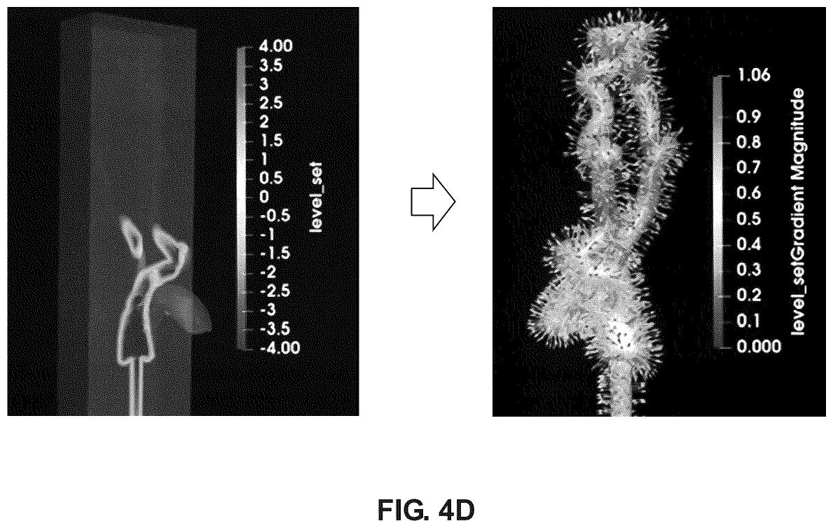

FIG. 4A illustrates a predictive control system according to one or more example embodiments.

The system includes an offline phase 405 and an online implementation phase 450.

The offline phase 405 may be considered pre-processing and training and the online phase 450 may be considered a real-time processing phase.

As shown, the offline phase 405 includes a data lake 410, a pre-processing module 415, an engine simulator 420 and an AI predictive control system 460.

The data lake 410 leverages available CTA images and physical parameters for devices. In some example embodiments, the data lake 410 may store physical parameters such as length of tip, shape and material for various types of guidewires, respectively, and other devices. Moreover, the data lake 410 may store patient data. For example, the data lake 410 may store 3D CTA brain and/or cardiac images for two planes.

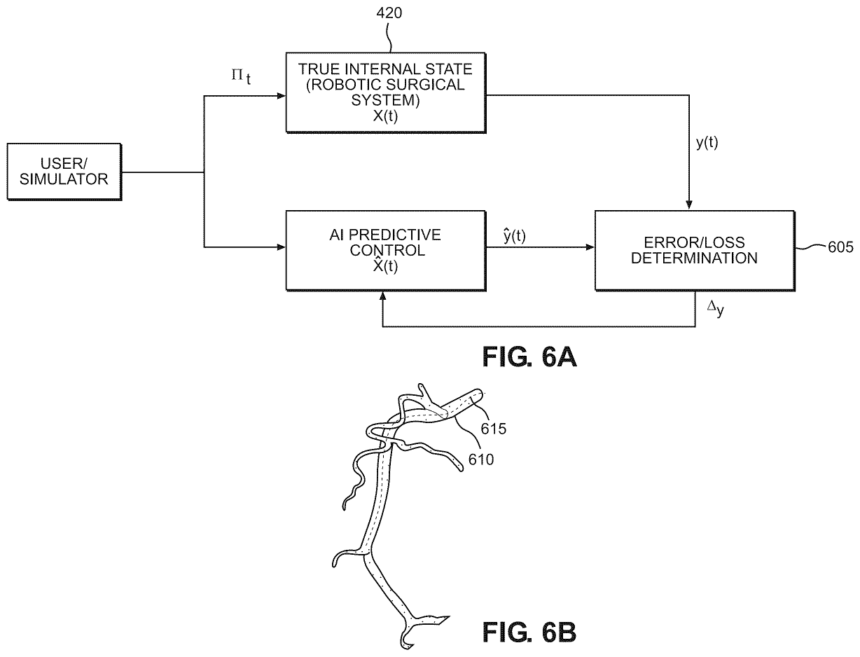

The pre-processing module 415 is configured to perform 3D vessel segmentation and extract a centerline (e.g., in FIG. 6B) with sectional information based on the CTA images from the data lake 410 and patient specific pre-op images 422 from an image processing module 480, which may be CTA images, but are not limited thereto. For example, the type of images and images may be adjusted such that the simulator 420 may determine set of motions for the guidewire as described with reference to FIG. 4B (e.g., 3D vessel segmentation, centerline determination and distance map generation).

The pre-processing module 415, engine simulator 420, the AI predictive control system 460 and the image processing module 480 and the functions thereof may be implemented by the controller 40. When the controller 40 is a processor, the processor may execute instructions stored in a memory 205 to perform the functions of the pre-processing module 415, engine simulator 420, the AI predictive control system 460 and the image processing module 480.

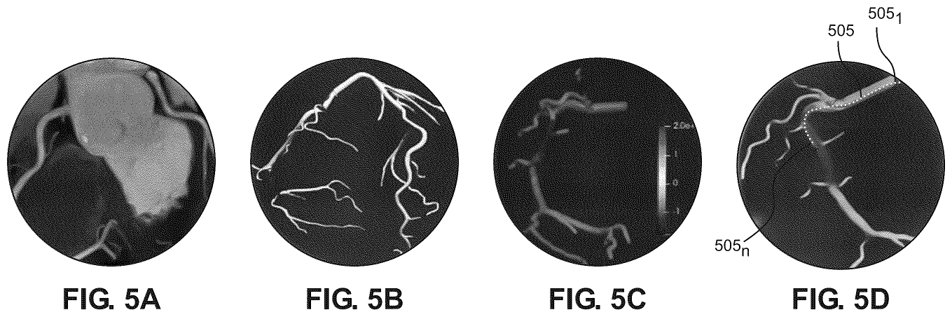

FIG. 4B illustrates a flow chart of generating a physics-based simulation of a guidewire and FIGS. 5A-5D illustrate images used to generate the physics-based simulation of the guidewire according to one or more example embodiments.

In some example embodiments, the pre-processing module 415 uses 3D CTA images of the patient (e.g., in FIG. 5A) and generates a corresponding 3D segmentation of whole coronary vessels (e.g., in FIG. 5B) at 415a. In some example embodiments, the segmentation may be performed using 3d U-Net as described at Tsang, Review: 3D U-Net-Volumetric Segmentation (Medical Image Segmentation), Towards Data Science, Apr. 2, 2019, the entire contents of which are incorporated by reference. At 415b, the pre-processing module 415 adds an insertion supporter (e.g., an introducer/sheath) of the guidewire as a boundary pipe, which is refined by a Poisson reconstruction filter to smooth irregularities in the reconstructed surface, making it more accurate and visually coherent within the overall 3D segmentation scene.

The pre-processing module 415 then computes a 3D level set distance map that contains a minimum distance to the vessels at each 3D point to define boundary conditions (e.g., in FIG. 5C) at 415c. In some example embodiments, the distance map may be generated according to Guo et al., DeepCenterline: a Multi-task Fully Convolutional Network for Centerline Extraction, arXiv:1903:10481v1, Mar. 25, 2019, the entire contents of which are incorporated herein by reference.

The pre-processing module 415 is further configured to generate a stenosis grading for angiography (i.e., for Fully Automated Coronary Evaluation (FACE)), which can be used as varied vessel lumen and friction information for the engine simulator 420. For example, the pre-processing module 415 may generate the stenosis grading as described in Avram, et al., CathAI: fully automated coronary angiography interpretation and stenosis estimation, npj Digital Medicine 6, Article number: 142 (2023), the entire contents of which are incorporated herein by reference.

The engine simulator 420 uses the information from the pre-processing module 415 (e.g., 3D vessel geometries, centerlines with section information, stenosis grading for frictional information, device properties) to create a physics-based simulation environment. The engine simulator 420 is configured to simulate in real time the motion of the guidewire in vessel geometries and can generate synthetic x-ray images based on the simulated guidewire motion.

Referring back to FIG. 4B, in an example embodiment, the engine simulator defines an initial position and direction of the guidewire at 420a. The guidewires may be simulated using a Cosserat-rod model. The engine simulator 420 applies a desired force to a proximal section of the guidewire using the Cosserat-rod model (at 420a), which interacts with the defined boundary conditions by solving partial differential equations. The Cosserat-rod model allows all possible degrees of freedom of deformation: bend, twist, stretch and shear. For example, FIG. 5D illustrates a guidewire 505 modeled by Cosserat-rod. As shown in FIG. 5D, Cosserat-rod models the guidewire 505 in n segments 505₁-505ₙ.

The guidewire is modelled as a deformable curve with attached deformable vectors to characterize its orientation. The orientation follows a system of coupled Partial Differential Equations to conserve both linear and angular momentum. In some example embodiments, a Neural Ordinary Differential Equation (ODE) approach is used to solve the partial differential equations efficiently to achieve real-time simulations, since the time integration using Neural ODEs is more accurate than using conventional time integrators. The inventors have discovered that Neural ODEs are useful to solve the conservation momentum equations from the Cosserat rod theory for guidewire insertion.

FIG. 4C illustrates a flow chart of generating a new mesh with a desired vessel-like structure to restrict the movement of a portion of a guidewire according to one or more example embodiments.

At S490, the simulator 420 obtains the 3D segmentation of the vessel structure of the patient (e.g., from the preprocessing). The simulator 420 uses a boundary condition for the guidewire to be kept inside the vessel while having the base of the guidewire restricted while it is being inserted. Thus, a new boundary condition is developed. The simulator 420 adds a vessel-like structure of a desired width to the entry point of large vessels at S492. Then, at S494, the simulator 420, cuts open meshes and finally applies a Poisson surface reconstruction filter is used to merge them in a watertight surface as shown at S496.

To restrict the catheter within the generated watertight surface, at each time step of the simulation, the simulator 420 computes a wall response that the vessels are applying to the rod (guidewire) when it gets close to the lumen wall, i.e.

ε=r−d>0 (1)

where d is the distance from the centerline of the guidewire to the closest point at the lumen and r is the radius of the guidewire. Then, the wall response force is given by

$${F}_{⊥}^{w} = ( - {F}_{⊥} + {k}_{w} ε - {v}_{w} v \cdot {u}_{⊥}^{w} ) {u}_{⊥}^{w}$$

where k_{w} is a stiffness of the wall, v_{w} is a dissipation coefficient, F_{⊥} is a sum of all rod forces against the wall, k_{w}ε is the elastic and

$${v}_{w} v \cdot {u}_{⊥}^{w}$$

is a dissipative term in normal velocity, where v is the applied velocity and

$${u}_{⊥}^{w} .$$

is a normal velocity perpendicular to a vessel wall.

The stiffness k_{w} and the dissipation coefficient v_{w} control the effect of the wall boundary condition.

To define this boundary condition, for each discretized point of the rod within the lumen, the minimal distance to the lumen's surface, as well as the normal direction may be computed. However, this operation is very time consuming and highly dependent on a number of points (size) of the lumen.

In some example embodiments, the simulator 420 uses a novel boundary condition, in which the watertight surface is used to compute a 3D image on a bounding box that contains the lumen mesh, which contains the level set map defined at each voxel as seen in FIG. 4D.

The level set map, shown in the left of FIG. 4D, is computed as a signed distance, so that its gradient is normal to the level set isocontours. The signed distance is computed as exact in a neighborhood of the surface, and can be extrapolated farther away, for efficiency of computation. Given any 3D point within the large vessel's lumen, the simulator 420 retrieves an approximation to its shortest distance to the lumen surface by just evaluating the 3D image of the level set map at that point, which is computationally efficient. As mentioned above, the normal direction is computed as well. Due to the definition of the level set map, this becomes straightforward by evaluating the gradient of the image at the desired point, as shown on the right of FIG. 4D.

Moreover, only one boundary condition is required, which avoids having to iterate over different boundary conditions defined at each point of the lumen's surface. This allows the simulations to be computationally efficient. Namely, for a lumen mesh with 17000 points, the application of this new boundary condition implies a speed-up factor of 3000 bringing the simulation to real-time. In real life, the vessels are continuously moving, making the guidance of the catheter more complicated. In example embodiments, the simulator 420 uses the level set method to move the vessel boundary, therefore having a continuously updated distance map that follows the vessel movement, which can be done using the boundary node method for cell motility as described in U.S. Pat. No. 10,282,638, the entire contents of which are incorporated by reference.

This provides the scalable simulated device states inside the vessels (e.g., in FIG. 5D). More specifically, the engine simulator 420 includes a set of device motions that can be applied in each created environment. The simulated device can consist of multiple segment points (e.g., one guidewire is defined as 300 segments points to model using the Cosserat-rod model). Each segment state can be defined as 3d position, velocity and applied force. The accumulated information of the segmented states indicate the device states.

The engine simulator 420 creates various states of the bedside system 12 using the device motions and associated environments as training data.

In addition, the simulator 420 may incorporate the motion of the guidewire into a Digital Reconstruction Radiography (DRR) algorithm to create synthetic X-ray images in real time. It should be understood that during the online phase, the AI predictive control system 460 and/or the image processing module 480 may also produce the synthetic images in the same or similar manner.

The motion of the guidewire at each exported time step may be integrated into the 3D CTA information by locating the closest voxels to the points of the guidewire and assigning them to have a maximum intensity. A rendering image with guidewire points with an assigned maximum intensity may be generated by the simulator 420. Once this information is merged, the simulator 420 uses a DDR algorithm, to simulate a C-arm and project the information onto 2D to generate synthetic X-ray images. The simulator 420 may use the DDR algorithm described in U.S. Pat. No. 10,282,638.

Finally, an output image is optimized using neural style transfer, by taking a set of ground truth images that can come from a fluoroscopic image or angiographic image of the patient, for example, which define the style reference image by blending it with the content image coming from the DRR.

The pre-processing module 415 and engine simulator 420 provide a scalable environment and associated various device controls in simulation which allows a reduced amount of CTA data and/or images of the patient during real device control.

The online phase 450 includes the trained AI predictive control system 460, the bedside system 12 and the image processing module 480. The AI predictive control system 460 and the image processing module 480 may be performed by the controller 40 or other hardware such as servers in the cloud.

The engine simulator 420 includes a set of device motions that can be applied in each created environment.

FIG. 6A illustrates a flow chart of training the AI predictive control system 460. As mentioned above, the various states of the bedside system 12 using the device motions and associated environments are used as training data. The AI predictive control system 460 may be trained off-line based on collected data from the pre-processing module 415, and the physics engine simulator 420. Patient-specific data can also be applied to the engine simulator 420 to validate and reinforce the AI predictive control system 460. The AI predictive control system 460 may be implemented as a recurrent neural network, long short-term memory (LSTM) network, a transformer architecture, a neural ordinary differential equations (ODE), other known deep learning architectures, a combination thereof or a sub-combination thereof. In some example embodiments, the AI predictive control system 460 may be implemented by the controller 40. In other example embodiments, the AI predictive control may be implemented by processing circuitry located remote from the bedside system 12 (e.g., in the cloud).

As shown in FIG. 6A, a user and/or simulator inputs a motion command πₜ to the robotic surgical system 12 and the AI predictive control system 460 for a time t. More specifically, the motion command πₜ is a command for the guidewire actuating mechanism 50 to advance/retract (translational movement) and rotate the guidewire and, thus, may also be referred to as a motor command or actuator command. In some example embodiments, the motion command πₜ may include at least one of a velocity or torque command for the guidewire.

The simulator 420 maintains a true internal state x(t) which may be a 3d-tip position of the guidewire at the time t from performing the motion command πₜ. More specifically, the AI predictive control system 460 is configured to predict a long-term state estimation based on traces that is the history of sequential states. In some example embodiments, the AI predictive control system 460 uses a recurrent neural network (e.g., RNN, LSTM, Transformer, N-ODE, etc.) to model a long-term state estimator.

The data generated from the simulator 420 is used to train the AI predictive control system 460. In some example embodiments, the input data from the simulator 420 can be a 3d-tip position, a centerline, a vessel friction, device properties, and a traveled distance at time t as well as the ground truth for the tip position (i.e., the true internal state x(t)). The AI predictive control determines a motion command π* based on our desired tip motion from the motion command πₜ.

The simulator 420 may determine an observed measured 3d position y(t) of the tip of the guidewire. The simulator 420 knows the tip position/applied torque (action) which creates a mapping function between the 3d tip position and the action.

In some example embodiments, the observed measured position y(t) may also be determined by the image processing module 480 using CTA images captured at the time t.

The true internal state may refer to the real observed value of the tip position of the guidewire, which may be observed using known sensors and/or models (e.g., from the simulator 420).

The AI predictive control system 460 maintains an estimated internal state \hat{x}(t) which may be an estimate 3d-tip position of the guidewire at the time t from performing the motion command πₜ. The AI predictive control system 460 outputs an estimated 3d position ŷ(t) of the tip of the guidewire. The estimated 3d position ŷ(t) of the tip of the guidewire in a vessel may be displayed in overlaid on the 3d vessel segmentation or a 2d x-ray.

At 605, the training system determines an error/loss Δy between the observed measured position y(t) and the estimated 3d position y(t) of the tip of the guidewire. The loss Δy is fed back to the AI predictive control system 460. As shown, FIG. 6A illustrates an AI neural network training process between ground truth (acquired from the simulator 420) and the AI predictive control system 460 (i.e., a target model). Thus, the AI predictive control system 460 is trained to be close to ground truth based on all the input conditions.

In some example embodiments, the AI predictive control system 460 is trained using mean-squared error (MSE) based on the error/loss Δy.

The AI predictive control system 460 may be generated by applying a sequence of 3d past tip position with vessel centerline, physical parameters, and traveled distance, and then creating a sequence of estimated states for future.

In some example embodiments, 100 CTA images for the right coronary artery are used as a simulator environment and 1000 randomly generated continuous sequential controls of π^{prox} are applied for 60 seconds, which generates 120 sequence sample points for each set of each sequential controls (one of the randomly generated continuous sequential controls) in each segmented vessel (one of the 100 CTA images). 80% of the data may be used as a training set and 20% may be used for a testing set. We then present the safety ratio and difference between the estimated force and the ground-truth in the test set.

FIG. 6B illustrates a 3D vessel geometry 610 with a centerline 615.

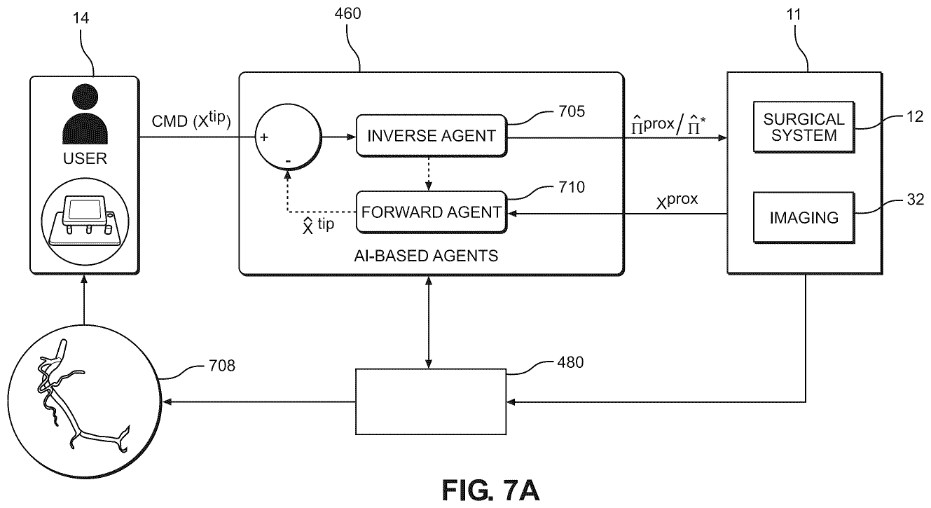

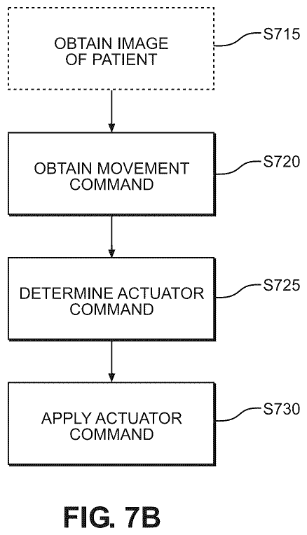

FIG. 7A illustrates a flow chart of an implementation of an AI predictive control system according to one or more example embodiments.

As shown, the AI predictive control system 460 includes an inverse agent 705 and a forward agent 710. The AI predictive control system 460 and the image processing module 480 may be performed by the controller 40 or other hardware such as servers in the cloud. In some example embodiments the inverse agent 705 and the forward agent 710 are trained as dual Transformer models such as described in Vaswani, et al., “Attention is all you need,” Advance in Neural Information Processing Systems, vol. 30, 2017, the entire contents of which are incorporated by reference.

More specifically, the inverse agent 705 generates a motion command of the guidewire \hat{π}^{prox} based on a difference between command position CMD and an estimated state of the tip of the guidewire \hat{X}^{tip} generated by the forward agent 710.

In some example embodiments, the forward agent 710 and the inverse agent 705 (i.e., forward/inverse transformer models) are learned with 30 sequences of states as inputs, 1 output (many-to-one), 12 heads, 4 encoder/decoder layers, 128 dimensions of feedforward, and 0.1 dropouts.

The inverse agent 705 may generate a motion command of the guidewire \hat{π}^{prox} (i.e., control of guidewire at connection to robot such as translation and rotation).

More specifically, x(t) is a 3d-tip position at time t, q(t) is motor state to apply at time t, e is vessel geometry (i.e., a lumen geometry from the level set distance map generated by pre-processing module 415) and a is one discrete action (i.e., a library of behaviors) at the proximal.

Given the current position of the tip of the guidewire x^{tip} and a desired location to go, the AI predictive control system 460 creates a sequence of motions minimizing an object cost (e.g., execution time, accurate motions) under constraints (e.g., no collision with a vessel and no puncture).

Thus, the AI predictive control system 460 uses reinforcement learning (RL) and includes the forward agent 710 for forward state estimation, which estimates a next state x(t+1) (e.g., position of tip of the guidewire), and the inverse agent 705 for inverse state estimation, which estimates a next action a(t+1) (e.g., at least one of torque or velocity).

At a guidewire's attachment point to the bedside system 12 (e.g., where a torquer is attached to the bedside system 12), a guidewire's proximal state is

x^{prox}≡ [symbol] Δ,φ [symbol] , (3)

where Δ is a translation and φ is a rotation.

At a distal (tip) of the guidewire, the distal state x^{tip} of the guidewire may be defined by its position and orientation in an SE (3) transformation which is a 3d homogenous transformation matrix consisting of translation and rotation.

Physical parameters p of the guidewire may include diameter, Poisson ratio, Young modulus and number of elements. However, example embodiments are not limited thereto. Let e∈E represent the spatial environment of the vessel, including 3D vessel geometry, 3D vessel centerline, and sectional labels.

Since the AI predictive control system 460 does not have a direct measurement for the tip of the guidewire, the AI predictive control system 460 simplifies x^{tip}∈ [symbol] ³ with regard to a 3D vessel centerline by projecting it into a known 3D vessel centerline (e.g., from the pre-processing module 415).

Therefore

x^{tip}≈\hat{x}^{tip}= [symbol] Δ^{tip},γ^{tip} [symbol] (4)

where Δ^{tip} and γ^{tip} represent the translation along the centerline and the distance from the centerline to the tip, respectively.

The forward agent 710 implements a forward transition function FF and the inverse agent 705 implements an inverse transition function IF as follows:

$${\hat{x}}_{t + 1}^{tip} = FF ( {\hat{x}}_{t}^{tip} , {x}_{t + 1}^{prox} , {π}_{t + 1}^{prox} ; p , e ) \qquad ( 5 )$$

$${\hat{x}}_{t + 1}^{prox} , {\hat{π}}_{t + 1}^{prox} = IF ( {\hat{x}}_{t + 1}^{tip} , {x}_{t}^{prox} , {π}_{t}^{prox} ; p , e ) \qquad ( 6 )$$

with control π^{prox}∈Π to transition from one state to another state given the parameters p and the spatial environment of the vessel e. The proximal states

$${x}_{t}^{prox}$$

(e.g., position before motion),

$${x}_{t + 1}^{prox}$$

(e.g., position after motion) are known from measurements of the guidewire actuator mechanism 50 (e.g., from motor encoder) and the proximal command

$${π}_{t}^{prox} and {π}_{t + 1}^{prox}$$

are also known from measurements of the actuator mechanism 50.

Given a desired tip state x^{tip} from the command CMD, and based on a current tip position (e.g., an observed tip position), the inverse agent 705 generates a sequence of proximal commands

$${\hat{π}}_{( t + 1 , ... , t + n )}^{prox}$$

for the surgical system 12 such that the surgical system manipulates the guidewire to a desired tip state

$${\hat{x}}_{( t + 1 , ... t + n )}^{tip} .$$

It should be noted, the inverse agent 705 also generates an estimated proximal position

$${\hat{x}}_{t + 1}^{prox}$$

(instead of measured position

$${x}_{t + 1}^{prox} )$$

to deliver proper commands to the bedside system 12.

The inverse agent 705 generates the sequence of proximal commands

$${\hat{π}}_{( t + 1 , ... , t + n )}^{prox}$$

by mining a cost C as follows:

$${\hat{π}}^{*} = \begin{matrix}arg min \\ {π}_{( t + 1 , ... , t + n )} ∈ π\end{matrix} C ( {π}_{t}^{prox} , {\hat{x}}_{( {( t + 1 , ... , t + n )}^{tip} \vert p} , e ) \qquad ( 7 )$$

where t is the current time and n is the long-term time step. More specifically, the AI control model 460 may be trained using mean-squared error (MSE) such that the AI control model 460 provides a sequence of actions minimizing the cost (e.g., errors).

Thus, the AI predictive control system 460 is configured to estimate a sequence of state information

$$( i . e . , {\hat{x}}_{( t + 1 , ... t + n )}^{tip} and {\hat{x}}_{( t + 1 , ... , t + n )}^{prox} , {\hat{π}}_{( t + 1 , ... , t + n )}^{prox} ) .$$

The image processing module 480 is configured to provide intermittent observation as well as provide an image illustrating the estimated states \hat{x}^{tip} and \hat{x}^{prox}. For example, the image processing module 480 (or the AI predictive control system 460) may generate an attention-based force map for the guidewire (or another percutaneous device) for display on one of the monitors 26 and 28. The attention-based force map may be generated by the image processing module 480 (or the AI predictive control system 460) in accordance with Li, et al, Efficient Self-supervised Vision Transformers for Representation Learning, ICLR 2022, arXiv: 2106.09785v2, Jul. 6, 2022, the entire contents of which are hereby incorporated by reference.

If a violation of the constraints is estimated by the AI predictive control system 460 (e.g., likelihood of guidewire contacting a vessel wall exceeds a threshold), the image processing module 480 provides an observation/measurement to the AI predictive control system 460. For example, the AI predictive control system 460 may compute uncertainty values based on errors between the centerline and the vessel wall, such that the AI predictive control system 460 can determine if the tip is in risk (i.e., too close to the wall in perpendicular direction). The AI predictive control system 460 can issue a notification to the user and/or the image processing module 480 to perform an observation from X-ray if the tip is determined to be close to the vessel wall.

In some example embodiments, the image processing module 480 uses a dynamic road map (DRM) and FACE stenosis grading with 2D device tracking information.

The 2D tracking information is registered by the image processing module 480 onto a 3D vessel lumen based on registration with an original CTA image to generate an actual measurement of the guidewire y(t). Then, the measurement y(t) can be applied into AI predictive control system 460 (to generate Δy) and reduce possible errors.

In addition, the image processing module 480 generates an image 708 which may reflect an estimated position of the guidewire and/or measured position of the guidewire permitting the user can supervise the procedure from uncertainty measures of AI predictive control. The image 708 can be also applied for autonomous decision-making. For example, given the parameters of the wire p and the spatial environment of the vessel e, the AI predictive control system 460 may provide a command to manipulate the wire to cross a stenosis, making all state transitions via control and finding a sequence of motions that minimizes cost C, defined as the completion time to cross target area or distance to vessel wall. The AI predictive control system 460 may calculate the sequence of motions

$$[ {π}_{1}^{*} , ... , {π}_{N}^{*} ]$$

using equation (7).

Thus, the AI predictive control system 460 generates a sequence of motion commands

$$[ {π}_{1}^{*} , ... , {π}_{N}^{*} ]$$

from a long worm predictive control. Furthermore, the cost function can be based on numerous factors such as completion time and safety permitting the AI predictive control system 460 to determine the sequence of motion commands

$$[ {π}_{1}^{*} , ... , {π}_{N}^{*} ]$$

in a real-time even without any observation. The optimal controls for a long-term horizon time will be applied to the robotic manipulator 12 to control autonomously or let the user confirm that controls to execute. One of advantage here is that if the safety boundary condition is not satisfied, then the predictive control can request new observation to the users, which allows the user to actively supervise the system or if it is autonomous system mode, then integrated image modality will be acquired new intermittent observation, as described above.

FIG. 7B illustrates a method of operation of the AI predictive control system of FIG. 7A. The method of FIG. 7B may be performed by the AI predictive control system 460 and, more specifically, the processing circuitry executing the AI predictive control system 460 (e.g., the controller 40).

As described with reference to FIG. 7A, the AI predictive control system 460 obtains at least one movement command for an intervention device of a robotic surgical system to a location within a patient (e.g., CMD) at S720. At S725, the AI predictive control system 460 determines at least one actuator command

$$( e . g . , [ {π}_{1}^{*} , ... , {π}_{N}^{*} ] )$$

for the robotic surgical system associated with the at least one movement command based on the movement command and an estimated current position of the intervention device. At S730, the AI predictive control system 460 applies the actuator command to the robotic surgical system.

Moreover, steps S720, S725 and S730 may be based on an image of the patient obtained at S715. For example, if the safety boundary condition is not satisfied, then the predictive control can request new observation to the users (e.g., new actual X-ray image), which allows the user to actively supervise the system or if it is autonomous system mode, then integrated image modality will be acquired new intermittent observation.

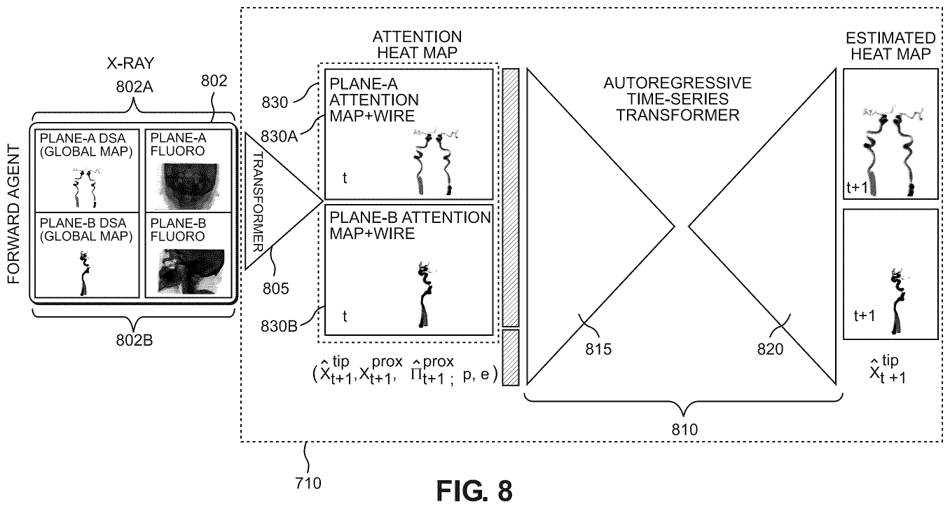

FIG. 8 illustrates a forward agent shown in FIG. 7A according to one or more example embodiments.

The forward agent 710 includes a transformer 805 and an autoregressive time-series transformer 810. The autoregressive time-series transformer 810 includes a long-term predictive encoder 815 and a long-term predictive decoder 820. The long-term predictive encoder 815 receives the estimated tip position,

$${\hat{x}}_{t}^{tip} ,$$

the proximal position

$${x}_{t + 1}^{prox} ,$$

the proximal state

$${π}_{t + 1}^{prox} ,$$

the estimated tip position

$${\hat{x}}_{t}^{tip}$$

physical parameters p and spatial environment e. Based on the input to the long-term predictive encoder 815, the long-term predictive decoder 820 generates the estimated tip position

$${\hat{x}}_{t + 1}^{tip} .$$

The transformer 805 is configured to generate at least one attention heat map 830 for image planes. The attention heat maps may be generated based on at least one of real images 802 generated by the imaging system 32 or estimated images from the AI predictive control system 460. In the embodiment shown in FIG. 8, the forward agent 710 receives a first plane image set 802a and a second plane image set 802a. Each of the images sets 802a, 802b may include a DSA image and a fluoroscopic image. The transformer 805 generates attention heat maps 830a, 830b for the first and second image planes, respectively. The long-term predictive decoder 820 is configured to generate estimated heat maps 835a, 835b for each image plane using the estimated tip position

$${\hat{x}}_{t + 1}^{tip} .$$

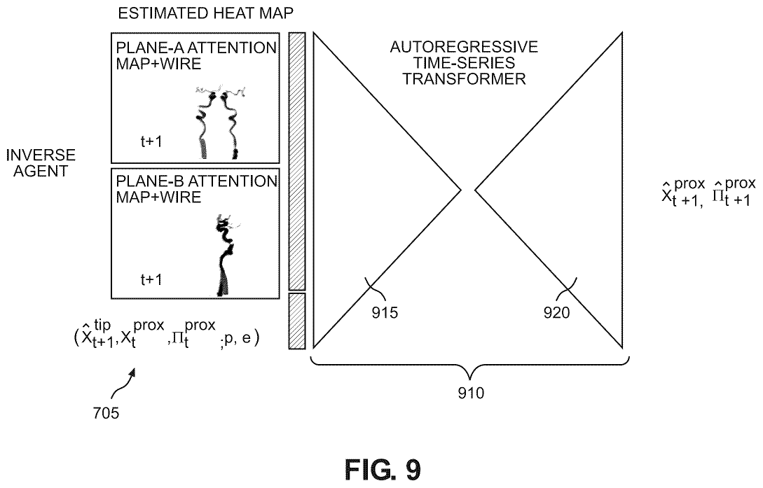

FIG. 9 illustrates an inverse agent shown in FIG. 7A according to one or more example embodiments.

The inverse agent 705 includes an autoregressive time-series transformer 910. The autoregressive time-series transformer 910 includes a long-term predictive encoder 915 and a long-term predictive decoder 920. The long-term predictive encoder 915 receives

$${\hat{x}}_{t + 1}^{tip} , {x}_{t}^{prox} , {π}_{t}^{prox} , p , e$$

as inputs.

As described, the AI predictive control system provides an efficient approach to indicate when to turn on/off X-ray.

In some example embodiments, the transformer models described herein may be the transformer described in Vaswani, et al., Attention Is All You Need, 31^{st} Conference on Neural Information Processing Systems (NIPS 2017), arXiv: 1706.03762v7, the entire contents of which are incorporated by reference.

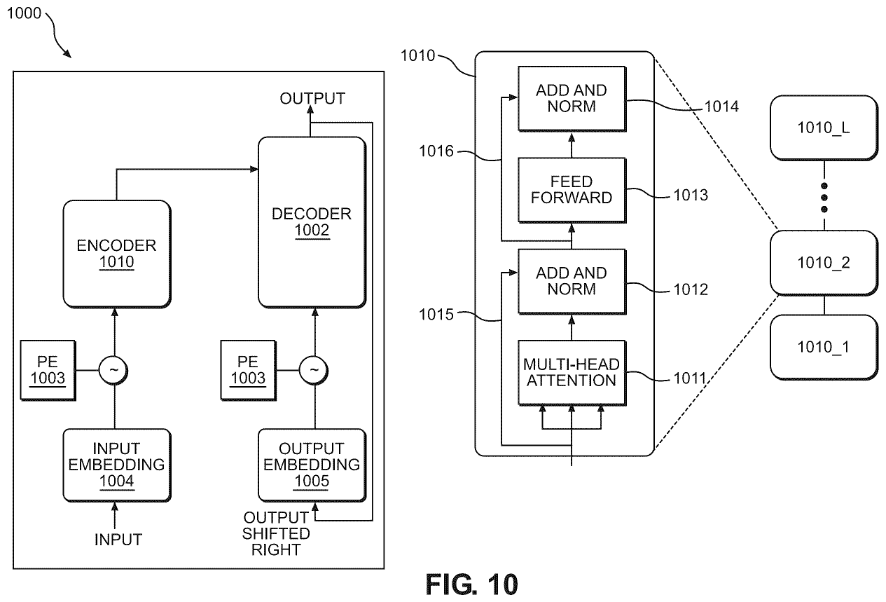

FIG. 10 illustrates an example transformer model used to implement the AI predictive control system according to one or more example embodiments.

As illustrated in FIG. 10, a transformer 1000 consists of an encoder 1010, decoder 1002, positional encodings 1003 with concatenation operation at the encoder 1010 and the decoder 1002, input embedding 1004, and output embedding 1005. An input x is embedded with positional encoding. The encoder 1010 maps an input sequence of symbol representations x to a sequence of representations. A multi-head attention mechanism 1011 performs self-attention processing on the inputs. The multi-headed attention mechanism 1011 encodes information about the relevant context, which allows the model to focus on relevant contexts at different length scales. A feed-forward network 1013 performs a post processing operation to generate the sequence of representations z. The decoder 102 generates an output sequence y of symbols one element at a time. The transformer 1000 uses L-layers in the encoder 1010, to perform sequential operations (e.g., 110_1, 110_2, . . . , 110_L) and L layers for the decoder 1002. Layers of encoder 1010 are processed by Add and Norm components 1012, 1014 via skip feeds 1015, 1016 to perform residual connection followed by layer normalization.

Although the terms first, second, etc. may be used herein to describe various elements, these elements should not be limited by these terms. These terms are only used to distinguish one element from another. For example, a first element could be termed a second element, and similarly, a second element could be termed a first element, without departing from the scope of this disclosure. As used herein, the term “and/or,” includes any and all combinations of one or more of the associated listed items.

When an element is referred to as being “connected,” or “coupled,” to another element, it can be directly connected or coupled to the other element or intervening elements may be present. By contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting. As used herein, the singular forms “a,” “an,” and “the,” are intended to include the plural forms as well, unless the context clearly indicates otherwise. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Specific details are provided in the following description to provide a thorough understanding of example embodiments. However, it will be understood by one of ordinary skill in the art that example embodiments may be practiced without these specific details. For example, systems may be shown in block diagrams so as not to obscure the example embodiments in unnecessary detail. In other instances, well-known processes, structures and techniques may be shown without unnecessary detail in order to avoid obscuring example embodiments.

As discussed herein, illustrative embodiments are described with reference to acts and symbolic representations of operations (e.g., in the form of flow charts, flow diagrams, data flow diagrams, structure diagrams, block diagrams, etc.) that may be implemented as program modules or functional processes include routines, programs, objects, components, data structures, etc., that perform particular tasks or implement particular abstract data types and may be implemented using existing hardware, for example, processing or control circuitry such as, but not limited to, one or more processors, one or more Central Processing Units (CPUs), one or more controllers, one or more arithmetic logic units (ALUs), one or more digital signal processors (DSPs), one or more microcomputers, one or more field programmable gate arrays (FPGAs), one or more System-on-Chips (SoCs), one or more programmable logic units (PLUS), one or more microprocessors, one or more Application Specific Integrated Circuits (ASICs), or any other device or devices capable of responding to and executing instructions in a defined manner.

Although a flow chart may describe the operations as a sequential process, many of the operations may be performed in parallel, concurrently or simultaneously. In addition, the order of the operations may be re-arranged. A process may be terminated when its operations are completed, but may also have additional steps not included in the figure. A process may correspond to a method, function, procedure, subroutine, subprogram, etc. When a process corresponds to a function, its termination may correspond to a return of the function to the calling function or the main function.

As disclosed herein, the term “memory,” “storage medium,” “processor readable medium,” “computer readable storage medium” or “non-transitory computer readable storage medium” may represent one or more devices for storing data, including read only memory (ROM), random access memory (RAM), magnetic RAM, core memory, magnetic disk storage mediums, optical storage mediums, flash memory devices and/or other tangible machine-readable mediums for storing information. The term “computer-readable medium” may include, but is not limited to, portable or fixed storage devices, optical storage devices, and various other mediums capable of storing, containing or carrying instruction(s) and/or data.

Furthermore, example embodiments may be implemented by hardware, software, firmware, middleware, microcode, hardware description languages, or any combination thereof. When implemented in software, firmware, middleware or microcode, the program code or code segments to perform the necessary tasks may be stored in a machine or computer readable medium such as a computer readable storage medium. When implemented in software, a processor or processors will perform the necessary tasks. For example, as mentioned above, according to one or more example embodiments, at least one memory may include or store computer program code, and the at least one memory and the computer program code may be configured to, with at least one processor, cause a network element or network device to perform the necessary tasks. Additionally, the processor, memory and example algorithms, encoded as computer program code, serve as means for providing or causing performance of operations discussed herein.

The terms “including” and/or “having,” as used herein, are defined as comprising (i.e., open language). The term “coupled,” as used herein, is defined as connected, although not necessarily directly, and not necessarily mechanically. Terminology derived from the word “indicating” (e.g., “indicates” and “indication”) is intended to encompass all the various techniques available for communicating or referencing the object/information being indicated. Some, but not all, examples of techniques available for communicating or referencing the object/information being indicated include the conveyance of the object/information being indicated, the conveyance of an identifier of the object/information being indicated, the conveyance of information used to generate the object/information being indicated, the conveyance of some part or portion of the object/information being indicated, the conveyance of some derivation of the object/information being indicated, and the conveyance of some symbol representing the object/information being indicated.

Benefits, other advantages, and solutions to problems have been described above with regard to specific embodiments. However, the benefits, advantages, solutions to problems, and any element(s) that may cause or result in such benefits, advantages, or solutions, or cause such benefits, advantages, or solutions to become more pronounced are not to be construed as a critical, required, or essential feature or element of any or all the claims.

### Non-Limiting Illustrative Embodiments

The following is a list of non-limiting illustrative embodiments disclosed herein:

Illustrative embodiment 1. A method comprising obtaining, by a trained system, at least one movement command for an intervention device of a robotic surgical system to a location within a patient; determining, by the trained system, at least one actuator command for the robotic surgical system associated with the at least one movement command based on the movement command and an estimated current position of the intervention device; and applying the actuator command to the robotic surgical system.

Illustrative embodiment 2. The method of illustrative embodiment 1, wherein the intervention device is a guidewire.

Illustrative embodiment 3. The method of illustrative embodiment 2, wherein the at least one movement command is a desired location within the patient of a tip of the guidewire.

Illustrative embodiment 4. The method of any one of illustrative embodiments 2-3, further comprising obtaining an observed position of the guidewire from the robotic surgical system; and determining the estimated current position of the guidewire based on the observed position.

Illustrative embodiment 5. The method of illustrative embodiment 4, wherein the observed position of the intervention device corresponds to an observed position of a proximal portion of the guidewire.

Illustrative embodiment 6. The method of illustrative embodiment 5, wherein the estimated current position corresponds to an estimated position of a tip of the guidewire.

Illustrative embodiment 7. The method of any one of illustrative embodiments 2-6, wherein the determining determines a series of actuator commands, each actuator command of the series of actuator commands corresponds to a state of the guidewire.

Illustrative embodiment 8. The method of any one of illustrative embodiments 2-7, further comprising generating image data displaying the estimated current position of the guidewire within a vessel of the patient.

Illustrative embodiment 9. The method of any one of illustrative embodiments 2-8, wherein the estimated current position of the guidewire is overlaid on a three-dimensional vessel segmentation.

Illustrative embodiment 10. The method of any one of illustrative embodiments 1-9, wherein the actuator command is at least one of a velocity command or a torque command for the actuator.

Illustrative embodiment 11. A trained system comprising processing circuitry configured to cause the trained system to obtain at least one movement command for an intervention device of a robotic surgical system to a location within a patient, determine at least one actuator command for the robotic surgical system associated with the at least one movement command based on the movement command and an estimated current position of the intervention device, and apply the actuator command to the robotic surgical system.

Illustrative embodiment 12. The trained system of illustrative embodiment 11, wherein the intervention device is a guidewire.

Illustrative embodiment 13. The trained system of illustrative embodiment 12, wherein the at least one movement command is a desired location within the patient of a tip of the guidewire.

Illustrative embodiment 14. The trained system of any one of illustrative embodiments 12-13, wherein the processing circuitry is configured to cause the trained system to obtain an observed position of the guidewire from the robotic surgical system; and determine the estimated current position of the guidewire based on the observed position.

Illustrative embodiment 15. The trained system of illustrative embodiment 14, wherein the observed position of the intervention device corresponds to an observed position of a proximal portion of the guidewire.

Illustrative embodiment 16. The trained system of illustrative embodiment 15, wherein the estimated current position corresponds to an estimated position of a tip of the guidewire.

Illustrative embodiment 17. The trained system of any one of illustrative embodiments 12-16, wherein the processing circuitry is configured to cause the trained system to determine a series of actuator commands, each actuator command of the series of actuator commands corresponding to a state of the guidewire.

Illustrative embodiment 18. The trained system of any one of illustrative embodiments 12-17, wherein the processing circuitry is configured to cause the trained system to generate image data displaying the estimated current position of the guidewire within a vessel of the patient.

Illustrative embodiment 19. The trained system of any one of illustrative embodiments 12-18, wherein the estimated position of the guidewire is overlaid on a three-dimensional vessel segmentation.

Illustrative embodiment 20. The trained system of any one of illustrative embodiments 11-19, wherein the actuator command is at least one of a velocity command or a torque command for the actuator.

Illustrative embodiment 21. A trained system comprising means for obtaining at least one movement command for an intervention device of a robotic surgical system to a location within a patient; means for determining at least one actuator command for the robotic surgical system associated with the at least one movement command based on the movement command and an estimated current position of the intervention device; and means for applying the actuator command to the robotic surgical system.

Illustrative embodiment 22. The trained system of illustrative embodiment 21, wherein the intervention device is a guidewire.

Illustrative embodiment 23. The trained system of illustrative embodiment 22, wherein the at least one movement command is a desired location within the patient of a tip of the guidewire.

Illustrative embodiment 24. The trained system of any one of illustrative embodiments 22-23, further comprising means for obtaining an observed position of the guidewire from the robotic surgical system; and means for determining the estimated current position of the guidewire based on the observed position.

Illustrative embodiment 25. The trained system of illustrative embodiment 24, wherein the observed position of the intervention device corresponds to an observed position of a proximal portion of the guidewire.

Illustrative embodiment 26. The trained system of illustrative embodiment 25, wherein the estimated current position corresponds to an estimated position of a tip of the guidewire.

Illustrative embodiment 27. The trained system of any one of illustrative embodiments 22-26, further comprising means for determining a series of actuator commands, each actuator command of the series of actuator commands corresponding to a state of the guidewire.

Illustrative embodiment 28. The trained system of any one of illustrative embodiments 22-27, further comprising means for generating image data displaying the estimated current position of the guidewire within a vessel of the patient.

Illustrative embodiment 29. The trained system of any one of illustrative embodiments 22-28, wherein the estimated position of the guidewire is overlaid on a three-dimensional vessel segmentation.

Illustrative embodiment 30. The trained system of any one of illustrative embodiments 21-29, wherein the actuator command is at least one of a velocity command or a torque command for the actuator.

1. A method comprising:
obtaining, by a trained system, at least one movement command for an intervention device of a robotic surgical system to a location within a patient;
determining, by the trained system, at least one actuator command for the robotic surgical system associated with the at least one movement command based on the movement command and an estimated current position of the intervention device; and
applying the actuator command to the robotic surgical system.

2. The method of claim 1, wherein the intervention device is a guidewire.

3. The method of claim 2, wherein the at least one movement command is a desired location within the patient of a tip of the guidewire.

4. The method of claim 2, further comprising:
obtaining an observed position of the guidewire from the robotic surgical system; and
determining the estimated current position of the guidewire based on the observed position.

5. The method of claim 4, wherein the observed position of the intervention device corresponds to an observed position of a proximal portion of the guidewire.

6. The method of claim 5, wherein the estimated current position corresponds to an estimated position of a tip of the guidewire.

7. The method of claim 2, wherein the determining determines a series of actuator commands, each actuator command of the series of actuator commands corresponds to a state of the guidewire.

8. The method of claim 2, further comprising:
generating image data displaying the estimated current position of the guidewire within a vessel of the patient.

9. The method of claim 8, wherein the estimated current position of the guidewire is overlaid on a three-dimensional vessel segmentation.

10. The method of claim 1, wherein the actuator command is at least one of a velocity command or a torque command for the actuator.

11. A trained system comprising:
processing circuitry configured to cause the trained system to,
obtain at least one movement command for an intervention device of a robotic surgical system to a location within a patient,
determine at least one actuator command for the robotic surgical system associated with the at least one movement command based on the movement command and an estimated current position of the intervention device, and
apply the actuator command to the robotic surgical system.

12. The trained system of claim 11, wherein the intervention device is a guidewire.

13. The trained system of claim 12, wherein the at least one movement command is a desired location within the patient of a tip of the guidewire.

14. The trained system of claim 12, wherein the processing circuitry is configured to cause the trained system to,
obtain an observed position of the guidewire from the robotic surgical system; and
determine the estimated current position of the guidewire based on the observed position.

15. The trained system of claim 14, wherein the observed position of the intervention device corresponds to an observed position of a proximal portion of the guidewire.

16. The trained system of claim 15, wherein the estimated current position corresponds to an estimated position of a tip of the guidewire.

17. The trained system of claim 12, wherein the processing circuitry is configured to cause the trained system to,
determine a series of actuator commands, each actuator command of the series of actuator commands corresponding to a state of the guidewire.

18. The trained system of claim 12, wherein the processing circuitry is configured to cause the trained system to,
generate image data displaying the estimated current position of the guidewire within a vessel of the patient.

19. The trained system of claim 18, wherein the estimated current position of the guidewire is overlaid on a three-dimensional vessel segmentation.

20. The trained system of claim 11, wherein the actuator command is at least one of a velocity command or a torque command for the actuator.

21. A trained system comprising:
means for obtaining at least one movement command for an intervention device of a robotic surgical system to a location within a patient;
means for determining at least one actuator command for the robotic surgical system associated with the at least one movement command based on the movement command and an estimated current position of the intervention device; and
means for applying the actuator command to the robotic surgical system.
